(19) United States
(12) Patent Application Publication
(10) Pub. No.: US 2014/0221843 A1
(43) Pub. Date: Aug. 7, 2014

(54) NEAR-INFRARED IMAGING FOR DIAGNOSIS OF SINUSITIS

(71) Applicant: The Regents of The University of California, Oakland, CA (US)

(72) Inventors: Joon You, Laguna Niguel, CA (US); Albert Cerussi, Rancho Santa Margarita, CA (US); Brian Wong, Irvine, CA (US)

(73) Assignee: The Regents of The University of California, Oakland, CA (US)

(21) Appl. No.: 14/171,446

(22) Filed: Feb. 3, 2014

Related U.S. Application Data
(60) Provisional application No. 61/760,035, filed on Feb. 2, 2013.

(51) Int. Cl. A61B 5/00

(52) U.S. Cl. CPC A61B 5/0086
USPC 600/473

(57) ABSTRACT

Disclosed herein are embodiments of a device and method for near-infrared imaging of a patients sinus cavities. The imaging can be used for diagnosis and monitoring of different conditions such as maxillary sinusitis. The system and method can use LEDs to provide near-infrared light through the tissue of a patient, where the light can be picked up by a camera. An iso-intensity contour line map can then be created for quantitative analysis.

### STATEMENT REGARDING FEDERALLY SPONSORED R&D

The disclosure described and claimed herein was made in part utilizing funds supplied by the National Institutes of Health under Grant No. 1R44DC012018 between the National Institutes of Health and the Regents of the University of California for the Small Business Innovation Research Grant. The government has certain rights in this invention.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

1. Field

Disclosed herein is a device and method for the diagnosis and management of sinusitis.

2. Description of the Related Art

In the United States, approximately one in seven people develop sinusitis each year, and 20 million cases of acute bacterial sinusitis become chronic to require medical treatment. Further, roughly 90% of adults have had sinusitis at some point in their lives. Sinusitis is an inflammation of the mucous membranes that line the paranasal sinuses. Sinusitis has many causes, such as infection, allergy, and autoimmune problems. Sinusitis can be classified into different categories such as: acute, recurrent, subacute, chronic, and acute exacerbation of chronic. All categories of sinusitis have generally the same systems, and can be hard to distinguish. Moreover, sinusitis can be differentiated by location in the sinuses such as amxillary, frontal, ethmoidal, and sphenoidal.

It is estimated over $3 billion is spent annually on all sinusitis related expenses. Furthermore, chronic sinusitis, one of the more prevalent chronic illnesses in the United States, generally evolves from unresolved acute sinusitis, and is a consequence of failure to accurately diagnose and provide complete therapy. Primary care physicians are the first to diagnose and treat up to 87% of these cases.

Current management of sinusitis is based mostly on observation of symptoms in primary care settings or radiation-based CT scans in specialist settings. Symptom-based observations do not provide consistent or standardized measure, and thus are completely ineffective for managing sinusitis. X-ray images are sometimes used but do not provide accurate results and cannot be used in pediatric populations. CT-scans are often used to confirm sinusitis diagnosis by ENT specialists but they are (a) too costly, (b) unnecessary in many cases, and (c) inappropriate for primary care settings.

Efficient management of chronic sinusitis remains a great challenge for primary care physicians. There is no simple, inexpensive, and safe method to accurately confirm the presence/extent of sinus disease. There is a great need for a simple office-based diagnostic technique that can reduce the time and cost related to under-treatments and unnecessary over-treatments (i.e. antibiotics) of chronic sinusitis.

### SUMMARY

Disclosed herein is a device for imaging a sinus cavity of a patient, the device can comprise a flexible illuminator configured to conform to the patient's upper palate, an optical output located on a distal end of the flexible illuminator, wherein the optical output is configured to direct radiation towards the patient's sinus cavity, an optical detector configured to receive the radiation from the optical output that passes through the patient's sinuses and out the patient's tissue, and provide a output signal, and an analyzer configured to create an iso-intensity line map from the output signal.

In some embodiments, the radiation can be near-infrared light. In some embodiments, the near-infrared light can have a wavelength of about 850 nm. In some embodiments, the optical detector can be a camera.

In some embodiments, the device can further comprise an optical diffusion layer located between the optical output and the patient's upper palate. In some embodiments, the optical diffusion layer can comprise a light scattering foam. In some embodiments, the optical output can be a plurality of LEDs. In some embodiments, the plurality of LEDs can be arranged in a bifurcated array.

In some embodiments,the analyzer can determine absolute light intensity levels along peak regions of iso-intensity lines, average intensity between a patients left and right sinuses, and asymmetry between left and right sinuses.

Further disclosed herein is method of imaging a sinus cavity of a patient, the method can comprise placing a flexible illuminator into the patient's mouth, wherein the flexible illuminator is configured to conform to the patient's upper palate, irradiating the patient's sinuses using optical sources located on a distal end of the flexible illuminator, receiving the radiation that passes through the patient's sinuses and out the patient's tissue, and analyzing the received radiation by determining an iso-intensity line map from the received radiation.

In some embodiments, the flexible illuminator can further comprise an optical diffusion layer located between the optical sources and the patient's upper palate. In some embodiments, the optical diffusion layer can comprise a light scattering foam. In some embodiments, the optical sources can be a plurality of LEDs. In some embodiments, the plurality of LEDs can be arranged in a bifurcated array.

Further disclosed herein is an at home self-diagnostic system for imaging a patient's sinus cavity, the system can comprise a stand configured to receive the patient's face so that the patient's face remains in a generally fixed position, a flexible illuminator configure to conform to the patient's upper palate, an optical output located on a distal end of the flexible illuminator, wherein the optical output is configured to direct radiation towards the patient's sinus cavity, an activator configured to activate the optical output, an optical detector located at a position to receive the radiation from the optical output that passes through the patient's sinuses and out the patient's tissue, and provide a output signal, and an analyzer configured to create an iso-intensity map from the output signal.

In some embodiments, the system can further comprise a display to display the iso-intensity map. In some embodiments, the system can further comprise an optical diffusion layer located between the optical output and the patient's upper palate. In some embodiments, the optical diffusion layer can comprise a light scattering foam. In some embodiments, the optical output can be a plurality of LEDs. In some embodiments, the plurality of LEDS can be arranged in a bifurcated array.

### BRIEF DESCRIPTION OF THE DRAWINGS

FIGS. 1A-B illustrate potential positioning locations of embodiments of a near-infrared sinus illuminator.

FIG. 2A illustrates an embodiment of a diffused NIR light captured by an NIR camera using ABS foam.

FIG. 2B illustrates an embodiment of an LED array mounted on a flexible mesh with a bifurcated array. The separation between left and right can be much wider to accommodate a wide separation between maxillary sinuses.

FIGS. 3A-C illustrate embodiments of a near-infrared imaging device.

FIGS. 4A-E illustrate embodiments of a near-infrared sinus illuminator.

FIGS. 5A-B illustrate flexibility of embodiments of a near-infrared sinus illuminator.

FIGS. 6A-D illustrate embodiments of LED arrays.

FIGS. 7A-B illustrate embodiments of a handle for a near-infrared imaging device.

FIG. 8 illustrates embodiments of a near-infrared imaging device.

FIGS. 9A-D illustrate embodiments of a near-infrared imaging device.

FIGS. 10A-B illustrate an embodiment of a method of use of near-infrared imaging device.

FIG. 11 illustrates an embodiment of a self-test sinus imaging system.

FIGS. 12A-B illustrate a comparison of old NIR images as compared to NIR images using embodiments of the disclosed device and method.

FIGS. 13A-B illustrate NIR images in (A) gray scale, and (B) in iso-intensity contour lines.

FIG. 14 illustrates contoured NIR images of various subjects using embodiments of the disclosure.

FIG. 15 illustrates an embodiment of a method for using a near-infrared imaging device.

FIG. 16A-B illustrate a patient after (A) and before (B) having a sinus infection on the right side.

FIG. 17A-B illustrate a patient having chronic sinusitis before (A) and after (B) use of oral steroids using embodiments of the disclosure.

FIG. 18 illustrates taking a reference image using an embodiment of the disclosure.

FIG. 19A-B illustrate normal subject NIR and CT comparison.

FIG. 20A-C illustrate a patient with mild/moderate sinus infection in (A) NIR, (B) CT, and (C) cross-sectional intensity plot.

FIG. 21A-C illustrate a patient with a severe sinus infection in (A) NIR, (B) CT, and (C) cross-sectional intensity plot.

FIG. 22A-C illustrate a patient with a moderate sinus infection in (A) NIR, (B) CT, and (C) cross-sectional intensity plot.

FIG. 23A-C illustrate a patient (A) before and (B) after undergoing a debridement procedure and a (C) cross-sectional intensity plot.

### DETAILED DESCRIPTION

Disclosed herein is a safe, non-invasive, comfortable and quantitatively consistent imaging device and method. In some embodiments, the device can be used to measure sinuses, for example maxillary sinuses, and diagnose sinusitis. Embodiments of the disclosed device can, for example, determine the presence of mucosal thickening and/or fluid buildup. Further, embodiments of the disclosed device can be used to monitor the progress of treatments of sinusitis.

Embodiments of the disclosed device and method can deliver a low-cost optical imaging tool for quick and simple assessment of sinusitis suitable for use by both primary care doctors and physician extenders. The device can use near-infrared (NIR) trans-illumination, and can create a complete digital image of a patient's sinuses at NIR wavelength spectrums. Further, embodiments of the disclosure can exploit the relative lack of light attenuation within living tissue and its potential sensitivity to the sinus structures and fluid characteristics. In some embodiments, the disclosed device and method can provide for fast data acquisition, patient comfort, and ease of use by non-technical personnel.

In some embodiments, a near-infrared imaging device can be composed of a handle and a NIR oral illuminator. A NIR oral illuminator can be used to light up a patient's maxillary sinuses in diffuse fashion to achieve a uniform illumination of both sinuses. NIR light can easily transmit through clear sinuses and out a patient's facial tissue, such as skin, and can be detected by NIR-sensitive digital cameras. Presence of fluid within sinus resulting from the infections can reduce the intensity of the light transmitted and also change the pattern of the transmitted light. Physicians can use varying intensity and spatial patterns to infer the health of the sinuses for diagnostic purpose as well as monitoring treatment progress. This disclosure can provide for a standardized method for managing sinusitis in primary care settings and enable cost-effective health care delivery by reducing over-and under-treatments.

Further disclosed are analysis methods for capturing and analyzing spatial patterns of NIR light transmitted through sinuses. The present disclosure can enable better quantitative management of sinusitis patients in primary care settings through the use of digital imaging. Further, disclosed herein are devices and methods that provide for consistent imaging and system calibration techniques. Accordingly, embodiments of the disclosed device and method are capable of obtaining reliable and consistent images, which is advantageous in diagnosing certain medical conditions, such as sinusitis.

In some embodiments, an intra-oral illuminator can conform to curvature variations in upper palate, and thus can help reduce the illumination errors. This can be accomplished by, for example, an illuminator design with bifurcated and flexible arrays of light sources and addition of light scattering media between the light sources and the tissues. In some embodiments, light emitting diodes (LEDs) can be used, though the type of light source is not limiting. Therefore, the disclosed device can create a uniform illumination on the surface of the upper palate with the optimal optical throughput. The uniform illumination can enable quantitative comparison between images as the differences in the images reflect the physiological or anatomic differences. Further, system calibration methods can enable quantitative comparison of the images based on their geometric configurations and intensity variations.

The terms “approximately”, “about”, and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

The present disclosure provides for a device and method for consistent image acquisition and quantitative analysis, thus allowing standardized diagnostic imaging of sinusitis in clinical settings. Further, the disclosed device and method can provide for advantages such as a comfortable and better fitting device allowing for quicker procedure time, faster acquisition time achieved through higher optical throughput from illuminator to the sinuses, more efficient light output allowing for faster imaging time (shorter shutter speed required to achieve the high signal to noise ratio), consistent illumination with reduced effects of anatomic variability allowing for quantitative comparison between various individuals and thus enabling better diagnostic criteria, more consistent illumination creating less errors due to anatomic variations in upper palate, and higher light throughput coupled with consistent illumination allowing for better quantitative comparison between normal vs. diseased leading to more accurate diagnosis.

### Diagnostic Device

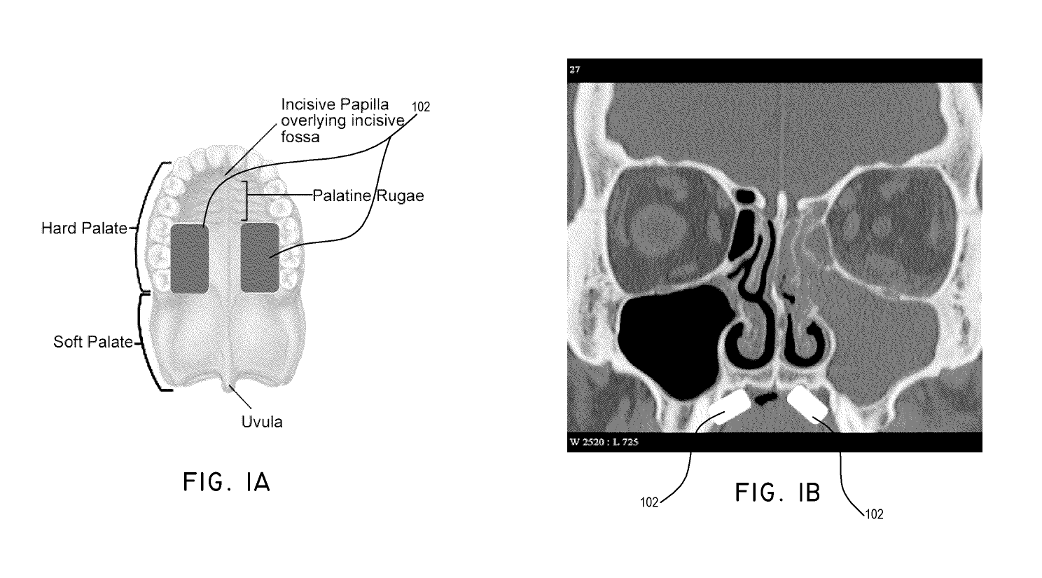

In some embodiments, an illuminator can be directed to a patient's upper palate in order to light up the patient's sinuses for further analysis. FIG. 1 shows an upper palate of a patient, along with a potential location 102 for embodiments of an illuminator. Illumination of the upper palate with embodiments of the disclosed device can be uniform which advantageously allows for the quantitative comparison of images between individuals and within the same individual.

The disclosed device can use optical outputs, such as, for example, LEDs, to direct light to a specific area of the sinuses, such as the maxillary sinuses. The LEDs can produce light in the NIR spectrum, such as at a wavelength of about 850 nm. In some embodiments, a single wavelength can be produced. In some embodiments, different wavelengths, or multiple wavelengths, can be used as well. The type and quantity of wavelengths are not limiting, and the wavelengths can be, for example, 700 nm, 800 nm, 900 nm, or combinations thereof. In a multi-spectral imaging setup, common biomedical spectroscopic methods can be applied to quantitatively determine the absorption and/or fluorescence of the intrinsic chromophores and fluorophores (i.e. fluid within sinuses, hemoglobin), or exogenous chromophores and fluorophores that have been introduced into the sinuses. For example, fluorescence of biofilm in middle ears may be used to distinguish between viral infection and bacterial infection. Such a differential diagnosis is critical for prescription of antibiotics since the use of antibiotics for viral infection is deemed ineffective.

In some embodiments, the light sources for sinus imaging can be implemented in the shortwave infrared (SWIR) range (wavelengths from about 0.9 to 1.7 microns). Further, in some embodiments the light sources can be in the forms of Light Emitting Diodes, lasers or other optoelectronic light sources, although the type of light source is not limiting. When SWIR light is used, detectors, such as Gallium Arsenide (InGaAs) sensors, can be used to provide physiological important information (i.e. water content) that can be useful in diagnosis of sinusitis.

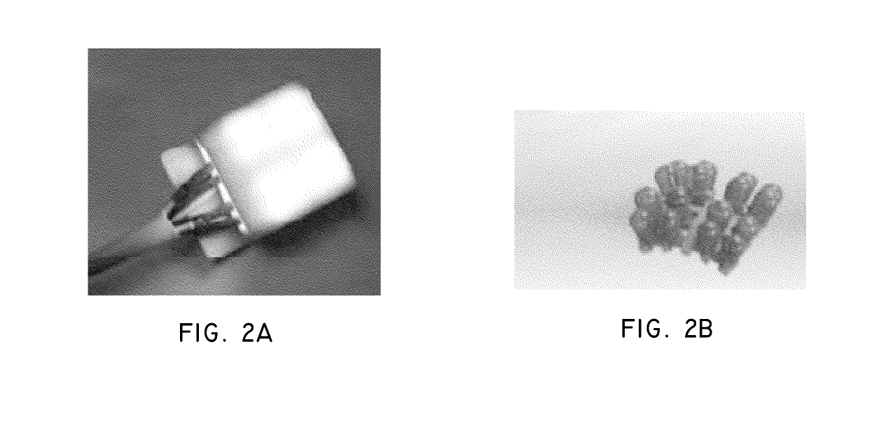

FIGS. 2A-B illustrate embodiments of LED lights which can be used in embodiments of the disclosure. However, using a simple bare LED arrays in contact with the upper palate can produce images that are highly sensitive to placement of the LEDs within a patient's mouth. As some LEDs make close contact with tissue while others don't, the illumination pattern can be highly sensitive to positioning of the illuminator. This can be due to the fact that the curvature of the upper palate varies widely among individuals and thus can make it difficult to create uniform light field when bare LEDs used. Accordingly, as further disclosed below, embodiments of the disclosed device can be flexible and thus can conform to a patient's mouth.

As shown in FIG. 2B, the LEDs in embodiments of the disclosure can be organized in an array that can be symmetrical between left and right side. This can be advantageous as left and right maxillary sinuses are separated by the nasal cavity and turbinates. When LEDs are placed in the centerline of the upper palate underneath the nasal cavity, the light field is very much prone to asymmetric illumination pattern as the center LEDs can be easily skewed to one side or the other. This sensitivity to the asymmetric illumination between left and right sides is mainly due to the fact that the middle of the upper palate sits below the nose rather than maxillary sinuses. Embodiments of the disclosed device can avoid direct illumination along the center-line of the upper-palate, thus avoiding possible asymmetric illumination resulting skewed placement of the LEDs between left and right sides of the upper palate. Also, bi-furcated design can enable better angle of illumination towards the maxillary sinuses rather than towards the nasal cavity. In some embodiments, no LEDs can be located in the center of the array. The extent of bifurcation (separation between left and right arrays) can be varied to accommodate for the size of the oral cavity and palate size. However, the bifurcated design is not limiting, and the LEDs could be located in a non-bifurcated arrangement.

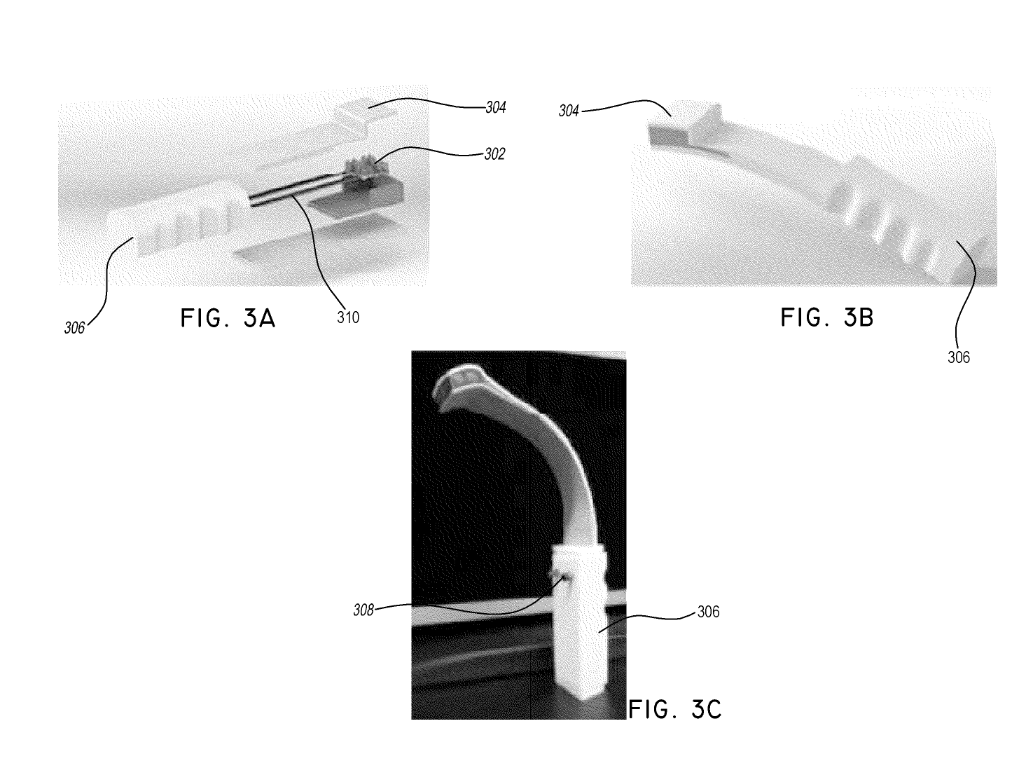

FIGS. 3A-C illustrate different configurations of embodiments of a device, which is described in detail below. As shown in FIG. 3A, in some embodiments the device can have LEDs 302 and an optical diffuser 304. The optical diffuser 304 can be located directly on top of the LEDs 302, and may or may not be in contact with the LEDs 302. Further, circuitry 306 can run from the LEDs 302 to a handle 306. As shown in FIG. 3C, the handle 306 can contain a switch 308 for activating the LEDs 302. Further, the switch 308 can change between different LED arrays as well as different wavelengths of light. In some embodiments, the handle 306 can contain a rechargeable battery for continuous use of the device. In some embodiments, constant current circuit can be used to keep the optical output from fluctuating wildly. Typical total power consumption for embodiments of an illuminator is about 100 mW. In comparison, typical LED flashlight emits power of 3-5 watts. In some embodiments, padding can also be added to the device to increase patient comfort. In some embodiments, higher optical power can be used. Higher optical power can be used to reduce the exposure time for capturing the NIR-based images. Short exposure time can help reduce light from the ambient lighting.

As mentioned, optical diffusers 304 that scatter light in near infrared spectrum can be applied as a filter for the illumination. Optical diffusers 304 can be made of, for example, foam or rubber, such as silicone rubber, thus allowing flexibility and comfortable fit. In some embodiments, EVA foam can be used. The foam can provide for structural enclosure, heat containment and can thus prevent user discomfort due to heat emanating from LEDs. The extent of diffusive illumination can be quantitatively tuned by using a rubber layers with optically scattering particles embedded in them. Such diffusers can be made from flexible tissue phantom materials such as, for example, silicone rubber and titanium dioxide powder. The addition of an optical diffuser 304 can reduce the uncertainties and variability of the illumination that can happen when bare LEDs or plastic-capped LEDs are placed in direct contact with the upper palate.

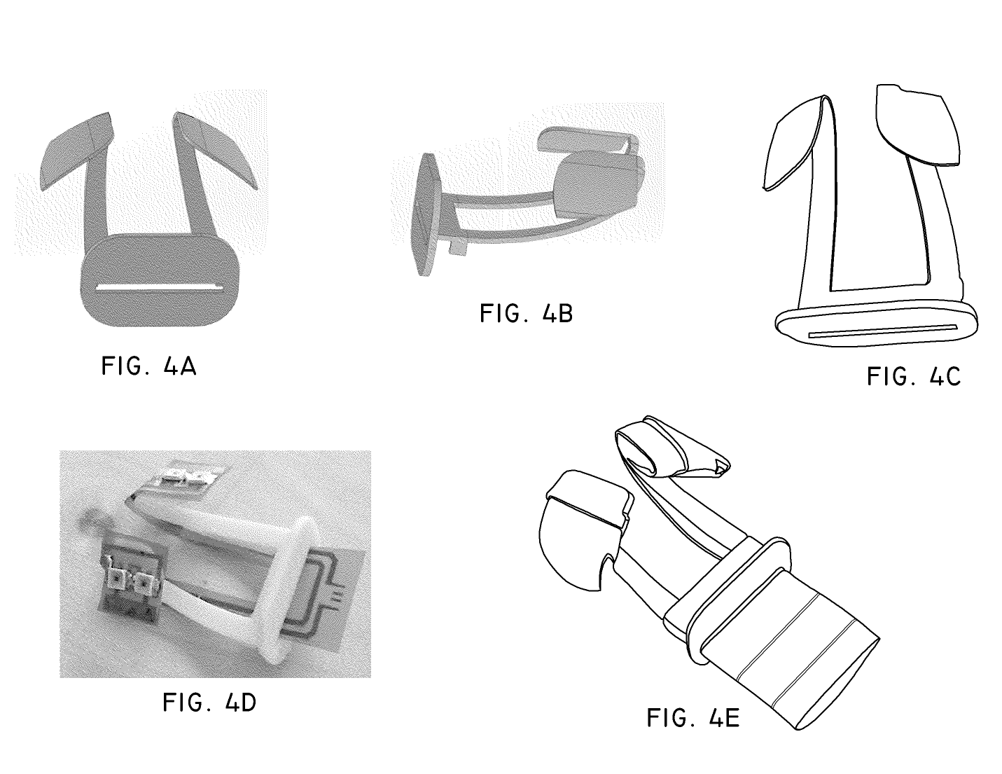

In some embodiments, as shown in FIGS. 4A-E, a sinus illuminator can be used to hold LEDs and attach to a handle. As mentioned above, LEDs can be positioned against a patient's hard palate, below molar/pre-molar teeth. Accordingly, embodiments of the disclosed illuminator can be made of a flexible material, such as metal sheet, plastic and/or rubber, though the material is not limiting, so that the illuminator can be pressed against a patient's hard palate comfortable. Further, as shown, the illuminator can have arms that can be configured to conform to the shape of the upper palate of a patient, thereby positioning the LEDS towards the sinus cavities in an optimal position. When a patient's mouth is closed with the illuminator in it, the LEDs can be naturally pressed up against the upper palate region above the molars and premolars, thus achieving the consistently good contact between the LEDs and the tissue.

Easy and consistent placement of the illuminator can be advantageous for practical deployment of embodiments of the disclosure in primary care applications. Accordingly, the illuminator can be comfortable to the patients, and may not require complex alignment or manipulation by the clinician or by the patients. Furthermore, the design of the illuminator can allow for consistent placement of the LEDs regardless of the patient's anatomic variability.

Previous illuminators used LEDs on a hard plastic base with a transparent plastic dome. The purpose of the transparent shell is to provide the maximum light input into the upper palate tissue surface while providing safe barrier between the mouth and the electronic components. However, this illuminator was quite bulky and did not conform to patients' anatomy, thus making the whole imaging experience uncomfortable. Further, hard shell illuminators do not provide consistent imaging quality as the placement of the illuminator is very much susceptible to the patient's anatomic variation in upper palate as the curvature can vary widely among individuals. Accordingly, as shown in FIGS. 3-4, embodiments of the disclosed device have addressed this issue by using, for example, a thin flexible LED arrays with a soft and optically diffuse filter layer placed between the LEDs and upper palates.

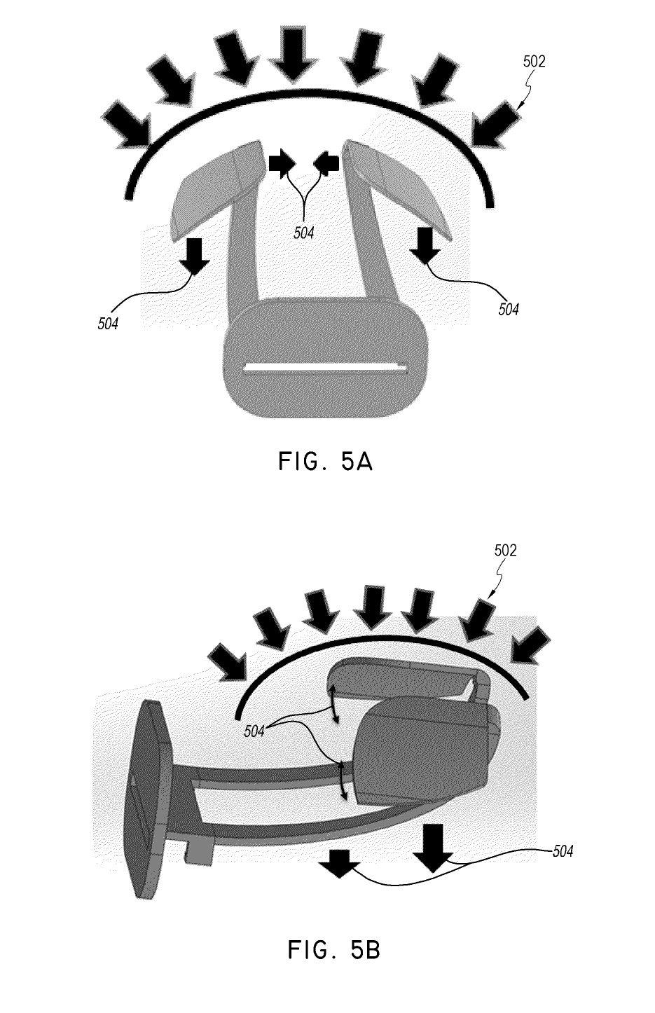

FIGS. 5A-B illustrate the flexibility of embodiments of a sinus illuminator that can allow it to conform to a patient's palate. As shown, when the illuminator is compressed by outside forces 502, the configuration of the illuminator can be compressed inwards, downwards, or generally in any direction 504 conforming to the outside forces 502. Thus, the sinus illuminator can fit and mold directly to a patient's palate.

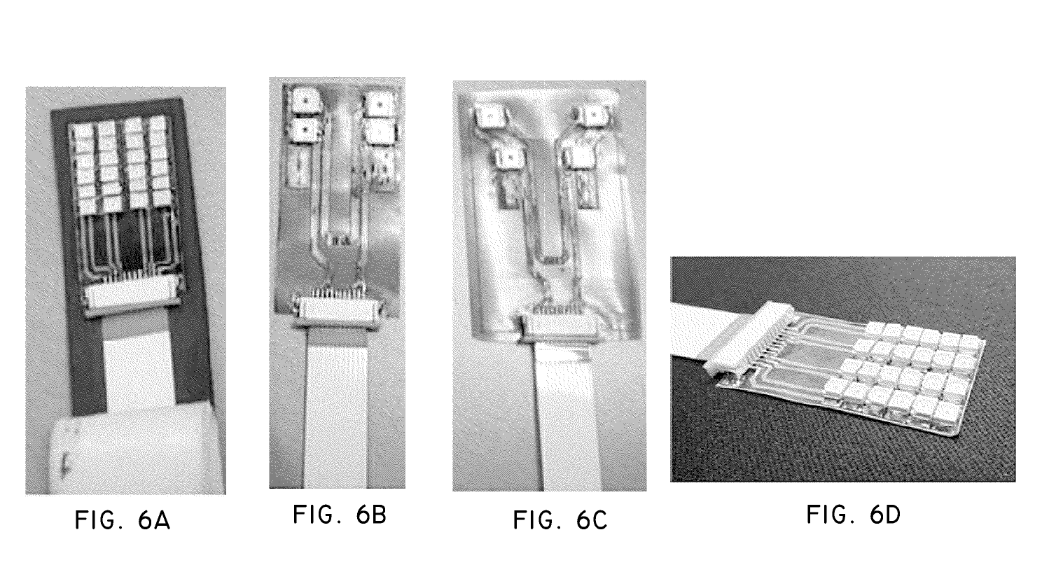

FIGS. 6A-D illustrate embodiments of circuitry for LED arrays, which can be built on, for example, a flexible base material to allow conforming to the curved surface of the upper palate. This can allow for consistent fitting of the whole LEDs to the upper palates regardless of the anatomic variations among individuals. It can also provide more comfort and more efficient optical coupling. Flexibility can be achieved by mounting LEDs on a flexible mesh, flexible circuit, rubber, or other flexible sheets.

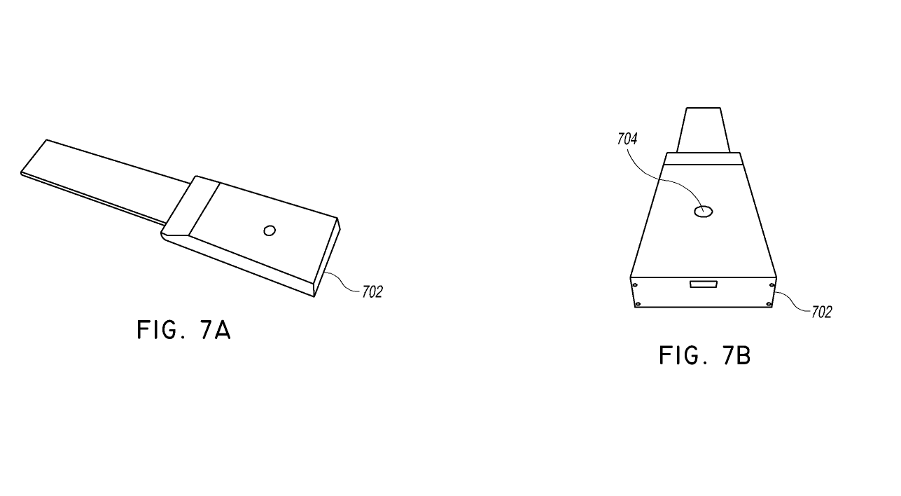

FIGS. 7A-B illustrate an embodiment of a handle 702 that can be used with embodiments of the disclosed device. As shown in the figure, the handle 702 can have a button 704 that can be used to operate the lighting system. However, other activation systems, such as a touch sensor, a switch, or voice activation, can be used as well, and the activation means is not limiting. Further, the handle 702 can contain a plurality of buttons to operate different procedures, such as NIR wavelengths or the use of multiple wavelengths.

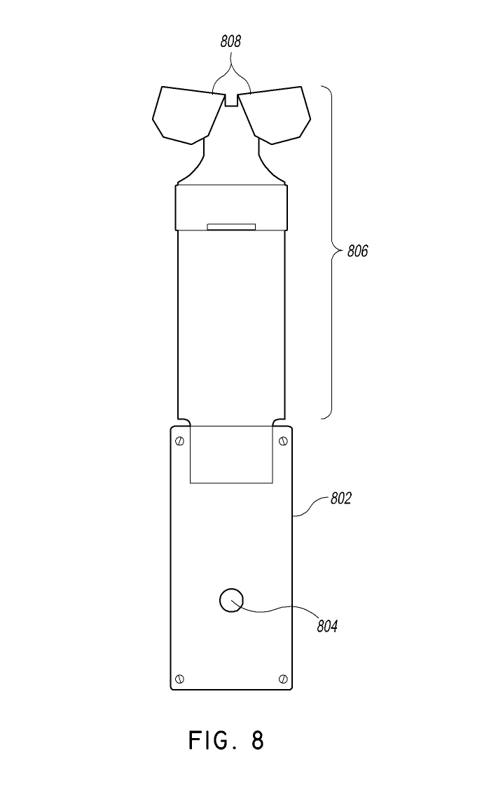

FIG. 8 illustrates an embodiment of a sinusitis diagnostic device. As shown, the device can have a handle 802 with a power button 804. This can be attached to a flexible illuminator 806, as described in detail above. The flexible illuminator 806 can have an optical output, such as LEDs, attached at the end distal 808 to the handle 802. In some embodiments, the LEDs can be incorporated directly into the illuminator 806. In some embodiments, a sanitary plastic bag can be used to cover the illuminator 806 and keep the illuminator from coming into contact with bodily fluid, which can allow for re-use of the illuminator 806.

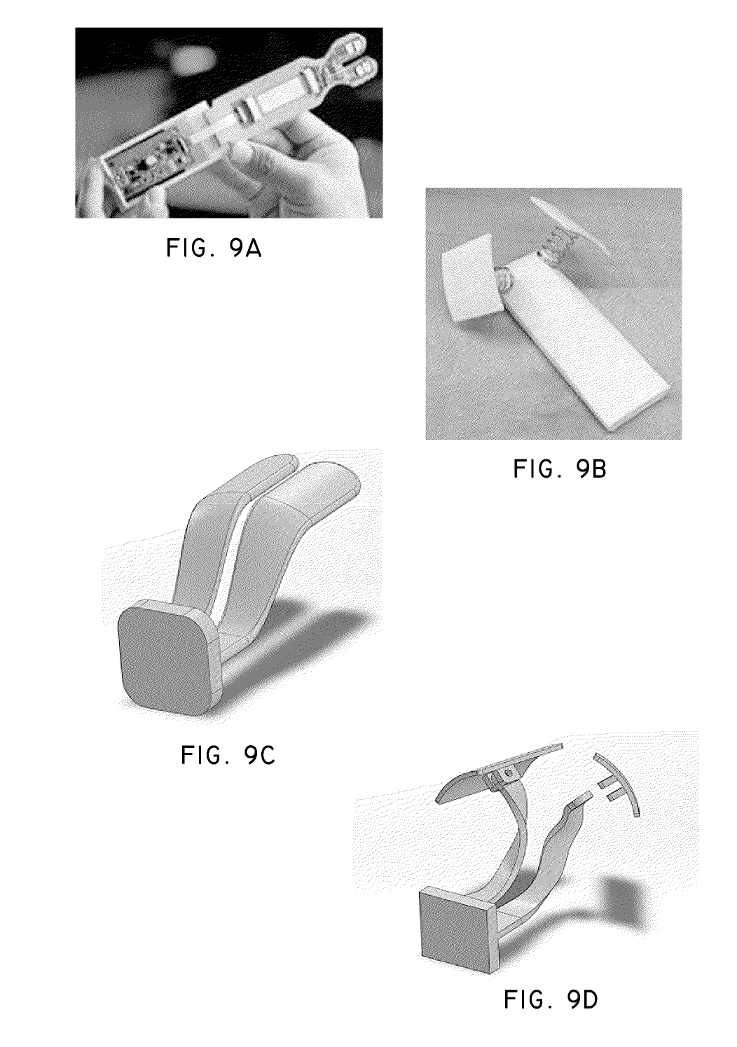

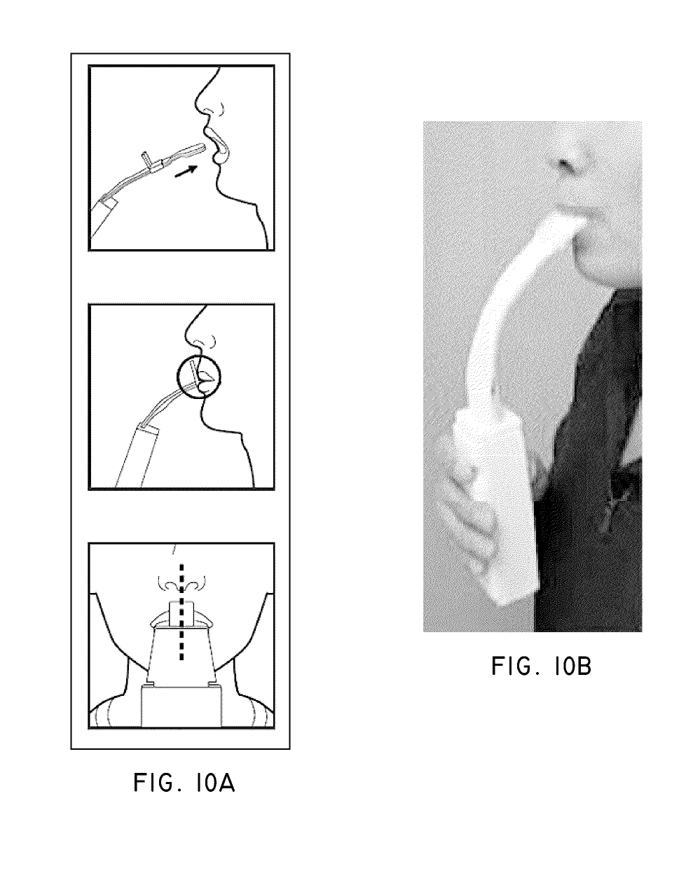

FIGS. 9A-D illustrate embodiments of the disclosed sinusitis diagnostic device. As shown in FIG. 9A, in some embodiments device can be generally flat with no protruding portions. Embodiments shown in FIG. 9B can use springs in order to mold the device to a patient's palate. FIG. 9C shows an embodiment with a flexible shape that can allow for natural fitting to a patient's upper palate when the patient's mouth is closed. FIG. 9D illustrates an embodiment with a hinge-based design. As shown, the end portions can be removable and replaced, and can rotate on an axis. Multiple sizes and configurations of the disclosed illuminators can be used to allow for fitting in different mouth sizes, such as infants and adults.

### Camera

In some embodiments, a digital camera can be used to capture the NIR light from the LEDs. The type of camera is not limiting, and any type of camera can be used. For example, a Canon S95 (point and shoot) or Canon T3 (DSLR) person camera could be used with embodiments of the disclosed method and system. A consumer digital camera can be advantageous as it does not require any additional training for new users, e.g. physicians, nurses, or patients. In some embodiments, the camera can be Wi-Fi compatible, and can automatically transfer data from the camera to, for example, mobile devices such as smartphones and tablets, and to another storage device, such as a computer server.

In some embodiments, the camera can be used with or without IR filters in order to limit the light to specific wavelength range. Further, the camera can be configured to capture images of all wavelengths. In some embodiments, the camera can have high ISO settings such as about 400, 800 or 1600, which can allow for fast data acquisition time (<½ second exposure). In some embodiments, the ISO settings of the camera can be around 12800-25600, although the ISO settings are not limiting.

In some embodiments, the camera can have a manual mode function to allow a customized approach for image acquisition settings. Further, the camera can have a low f number for capturing images in low light condition.

In some embodiments, the built-in camera of mobile devices such as smartphones, tablets and laptops can be used to allow easy capture and review of the images even in home settings. In such devices, the illumination trigger, image capture, display and storage can be accomplished within a single device. In some embodiments, sinus illumination and image capture can be synchronized electronically via wire or optically via IR sensor between camera and illuminator.

### Method of Use

In some embodiments, for measuring the health of sinuses, a near-infrared oral illuminator, as discussed in detail above, can be inserted into a patient's mouth with the light sources placed towards the upper-palate region beneath the maxillary sinuses. The patient or a doctor/nurse can push the illuminator to make a snug contact with the upper palate of the mouth. The illuminator can then be turned on, for example by a patient or physician turning a switch, to light up the sinuses for 1-10, 1-5, 2-5, or 2-3 seconds to allow image acquisition. A near-infrared sensitive digital camera is used to capture the light that has transmitted through the patient's sinuses and cheeks. Images of the face, particularly in the cheek region can be acquired using a near-infrared sensitive camera. The general insertion of embodiments of an illuminator into a patient's mouth can be shown in FIGS. 9A-B.

In some embodiments, images taken by the camera can be uploaded to a general computing database such as, for example, cloud-computing. For example, when an image is acquired at a clinic, it can be wirelessly transferred to the server (on-site or off-site) where it can be processed via various imaging algorithms and the final results can be transmitted back to the user and displayed on a mobile computing device, such as a tablet or laptop. Further, an imaging database can be formed that allows a patient or physician to compare their images with other images.

Spatial pattern of the transmitted light is then used to infer the health of the sinuses (i.e. presence of fluid, or inflammation and anatomy anomalies). Embodiments of the disclosed illuminator can also be composed of multiple wavelengths to achieve tissue spectroscopy and potentially separate the absorption spectra of the fluid or biofilm inside the sinuses using tissue spectroscopy techniques such as diffuse optical spectroscopy. The temporal behavior of the sampled tissue can also be assessed (i.e., pulsatile signals).

In some embodiments, tomographic reconstruction of the sinus structures can be achieved by selectively switching different LEDs and capturing multiple images using a camera system with fixed angle relative to the front of face. In some embodiments, tomographic imaging can be achieved using multiple camera angles for a fixed illumination pattern or in combination with multiple illumination patterns.

### At-Home Self-Testing Diagnostic Device

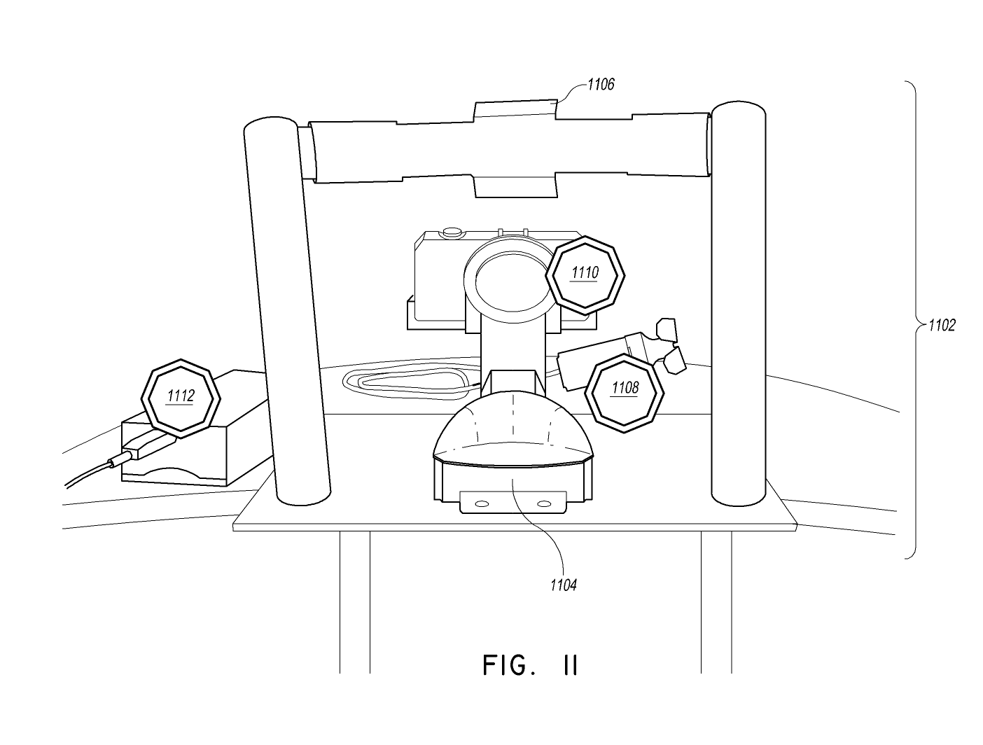

In some embodiments, the disclosed device can be used with an integrated system for self-testing as shown in FIG. 11. The system can contain a holder 1102 for a patient's head/chin. For example, a chinrest 1104 can be used along with a bar 1106 where a patient can rest their head.

The system can further include a mouthpiece 1108 and camera 1110 which can be adjusted to the correct positioning. The patient can then simultaneously turn on the LED of the mouthpiece 1108 and take a picture with the push of a single button of a trigger box 1112. The button can operate the LEDs and camera 1110 simultaneously. In some embodiments, two buttons can be used to operate the devices separately. In some embodiments, the camera 1110 can display the NIR images. In some embodiments, the system can further have a display device connected to the camera 1110, so that the patient can see the NIR images. Thus, no physician may be needed during use of the system and a patient can test themselves in the comfort of their own home. In some embodiments, the self-testing diagnostic device can be used in less than 10, 5, 4, 3, 2, or 1 minutes.

### Quantitative Imaging

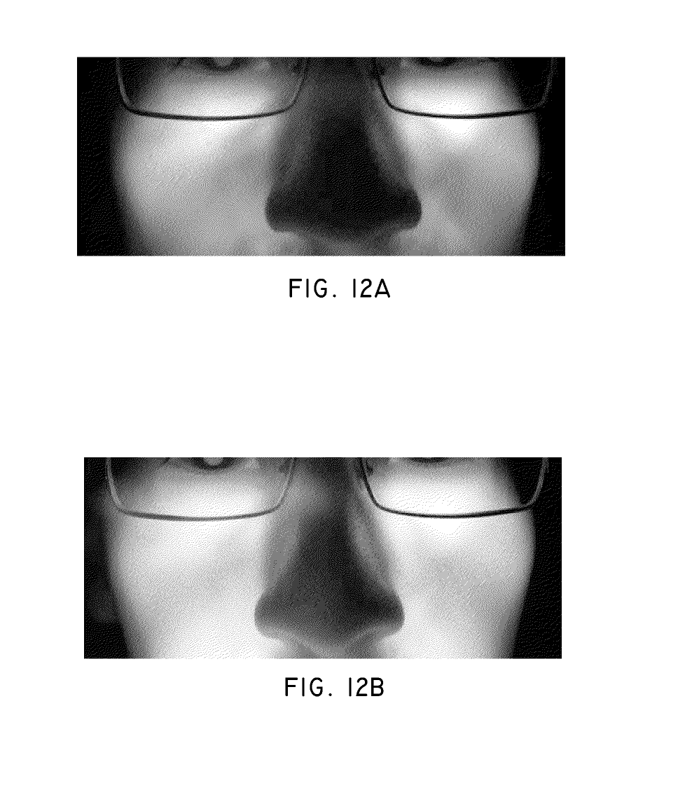

In embodiments of the disclosed device, it is possible to distinguish the subtle differences among individuals and within an individual and found correlation to the subjects' sinus health. Embodiments of the disclosed device provide for significantly more NIR light than previous devices. This is shown in FIGS. 12A-B, where FIG. 12A shows previous lighting methods, and FIG. 12B shows the amount of light in embodiments of the disclosed device. This higher lighting capability disclosed herein can allow imaging of lower intensity light fields with lower noise, thus providing better signal to noise ratio for low-light sinus imaging.

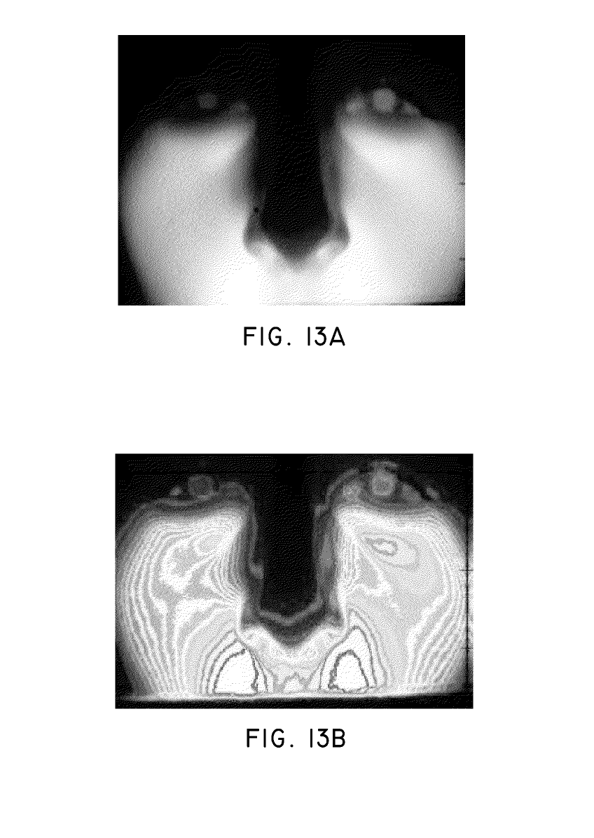

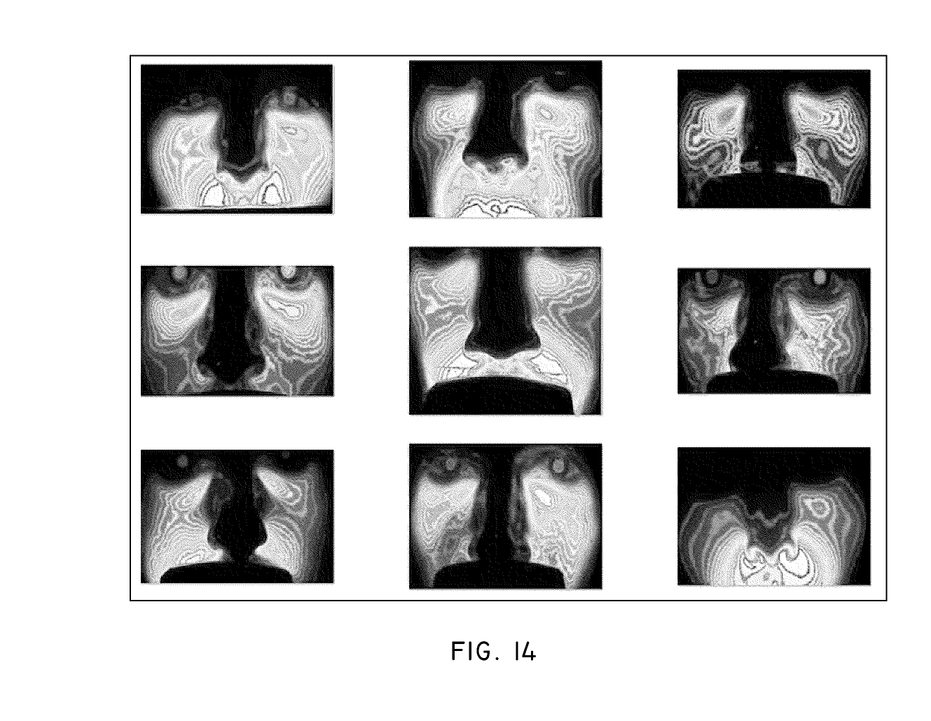

One method of quantitatively diagnosing or monitor sinusitis is through the use of forming iso-intensity lines (e.g. lines created by the same intensity pixels). This method can allow for easily comparing two images. The quantitative use of embodiments of the disclosed method is evident when comparison is made between the gray scale image to iso-intensity line images in FIGS. 13A-B. Use of iso-intensity line analysis can allow one to perform complex geometric analysis of the light patterns and thus enable subtle quantitative comparison between normal and diseased sinuses. The geometric shape variability of the NIR sinus images becomes apparent when one examines the various intensity pattern shapes among individuals. NIR images of 9 ‘normal’ volunteers' are shown in the FIG. 14. They all display intensity patterns that coincide with the location of their maxillary sinuses. The variations in the patterns may be due to anatomic diversity or partial fluid build-up without symptoms. It is notable that the last two subjects have somewhat asymmetric image patterns. The center bottom figure belongs to a subject that has frequent sinus infections on his right side maxillary sinus. The bottom right subject has been recuperating from a sinus infection on her right sinus for the last 20 days. When comparing to the NIR sinus images of 20 days ago, subtle differences can be seen that may be results of sinus fluid clearing. The subject had shown improvement in symptoms over the 20 days but had not completely recovered back to normal state at the time.

Iso-intensity maps, or contour maps, are a graphing technique used in engineering, mathematics, meteorology, physical geography and oceanography. Each curve can connect points where the pixel has the same particular value. By defining multiple values, multiple contour lines can be drawn in a 2 dimensional map showing the gradient of the intensity, which is perpendicular to the contour lines. When the lines are close together the magnitude of the gradient is large, meaning the intensity drop is steep. The intensity difference between each adjacent level of contour lines can be set to be linear (i.e. increasing at a constant step) or nonlinear (e.g. exponential). Contour maps can be generated by registering and displaying the positions of pixels whose intensity match the specific intensity level or range of intensity defined by the user.

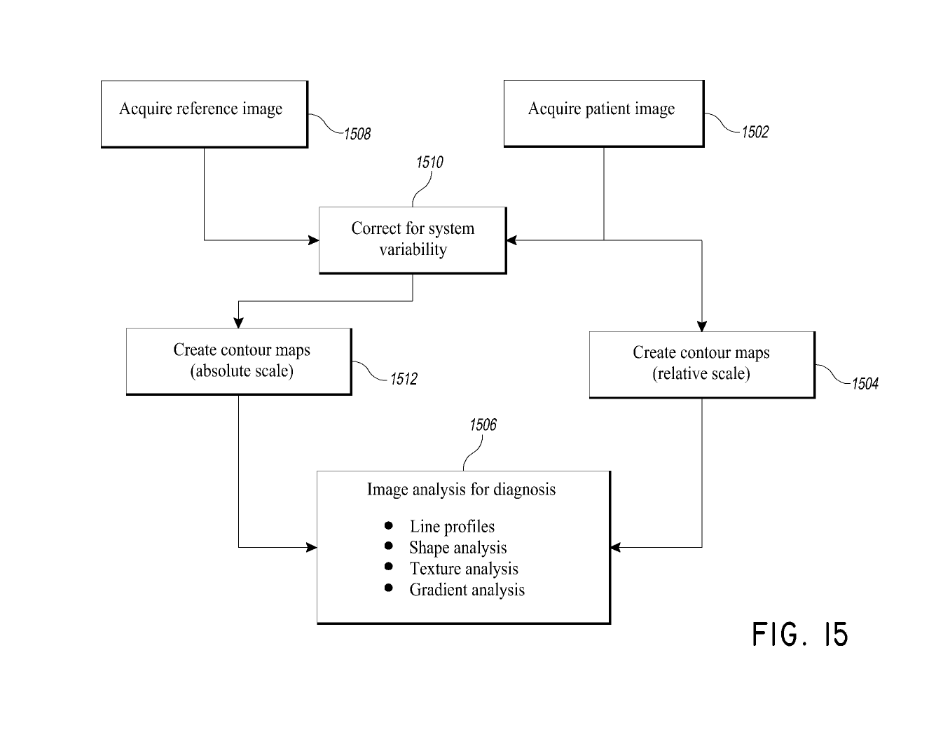

In embodiments of the disclosure, iso-intensity lines can be generated by using a relative scale for each image or by using an absolute intensity levels determined with a calibration process. Embodiments of this are shown in FIG. 15. First, an image of a patient can be acquired 1502, as described in the disclosure. In a relative scaling 1504, levels of intensity may be defined by dividing equally the whole dynamic range of intensity for each image. For example, for a typical 8-bit JPEG digital image which has 256 discrete intensity levels, the intensity may be equally divided into 10 discrete levels based on 256 intensity levels observed. In an absolute scaling 1512, each intensity level observed in an image can be converted to an absolute scale by comparing the sample image to the calibration image. Typical calibration images can be taken 1508 with a diffuse media with known light source intensity, further described below in the intensity balancing. The absolute scale 1512 can correct for system variability 1510. Regardless of the scale used, an image analysis for diagnosis 1506 can be obtained. The image analysis can be, for example, line profiles, shape analysis, texture analysis, or gradient analysis. [0064] In some embodiments, geometric analysis of the transmitted light pattern based on iso-intensity distribution can be used to determine such quantitative parameters. For example, absolute intensity level along the peak region, average intensity between left and right sinuses, and asymmetry between left and right sinuses can all be analyzed.

Further, various illumination patterns can generate dramatically different shapes of light pattern only when revealed with iso-intensity light. These shape patterns can reveal the anatomic structure and physiology of a patient's sinuses. For example, thin bone layer in the cheekbone can transmit more light than thicker ones. Also, fluid accumulation at the bottom of the maxillary sinuses can reduce the general intensity of the light field while fluid or biofilm build-up on upper part of the maxillary sinuses can change the shape of the light pattern differently from the normal subjects. Accordingly, shapes can be categorized into a typical shape patterns based on multiple parameters such as, for example, distance of the peak intensity position relative to the eyes/nose, and geometric shape (oval, skewed triangle, circular, etc.).

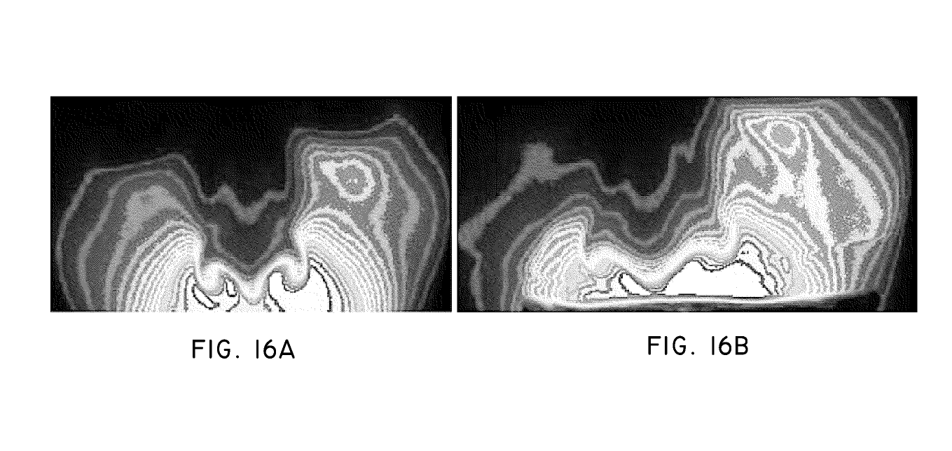

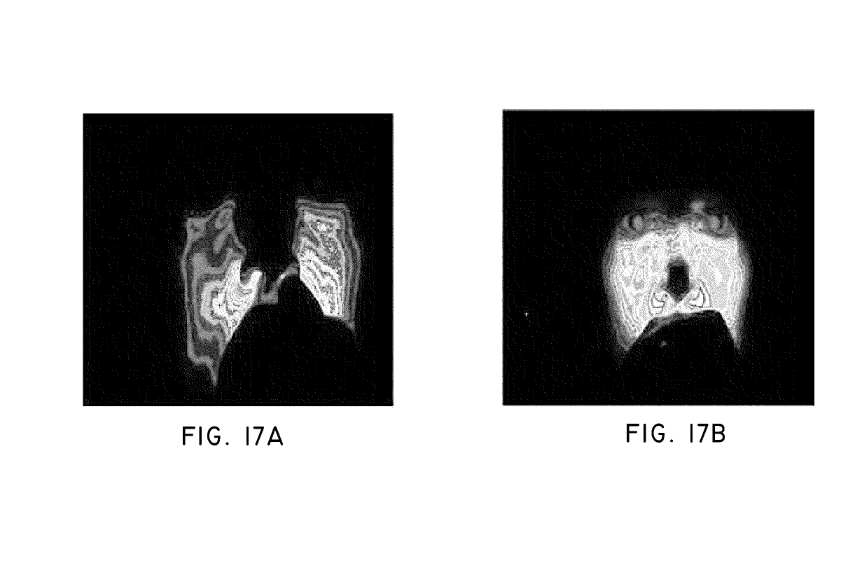

FIGS. 16 and 17 illustrate different quantitative uses of the disclosed device and method. FIGS. 16A-B illustrate a patient's NIR results after (FIG. 16A) and before (FIG. 16B) a sinus infection. FIG. 17A was taken when a patient came in with nasal obstruction and turbinate hypertrophy indicating chronic sinusitis. FIG. 17B was taken after the patient has undergone oral steroids and has experienced substantial improvement in the symptoms.

### Intensity Balancing

LEDs optical power output as well as the cameras sensitivity can vary with the ambient temperature, internal heating, aging, and battery power, as well as optics (lens/zoom). In order to compare images between individuals or the same individual at different times, this variability should be accounted for.

An intensity balancing scheme can be used to reduce and eliminate the sensitivity variability between images. The intensity balancing method is similar to the white balancing of color images used in digital color cameras. White balancing of a color image can be used when a photographer wants to eliminate the effect of the ambient lighting on the color of an object being photographed. White balancing thus reduces and eliminates the color variation of an object when its ambient lighting condition changes. Typically, a photographer takes a photo with white or gray color reference sheets and uses this ‘reference’ image to balance out the sensitivity between red, white and blue channels in the digital image sensor. Auto white balance is available in all digital cameras and uses a best guess algorithm within a limited range.

In some embodiments, a custom white balance can allow a patient and/or doctor to take a picture of a known gray/white reference under the same lighting, and then set that as the white balance for future photos. In intensity balancing for sinus transillumination, an image can be acquired using a reference object that provides both diffuse scattering and relatively flat spectral absorption profile in the wavelength range of interest (i.e. 600 nm-1500 nm). By taking reference images for multiple exposure time for each wavelength of LED being used, the reference images can be generated for particular set of camera and LED illuminator. These reference images can then be used to determine the variation of the imaging system between each imaging session, as any intensity variation in the image of the reference object is due to the system variability. For example, any changes in the intensity of the reference object between two imaging session may result from the system variability.

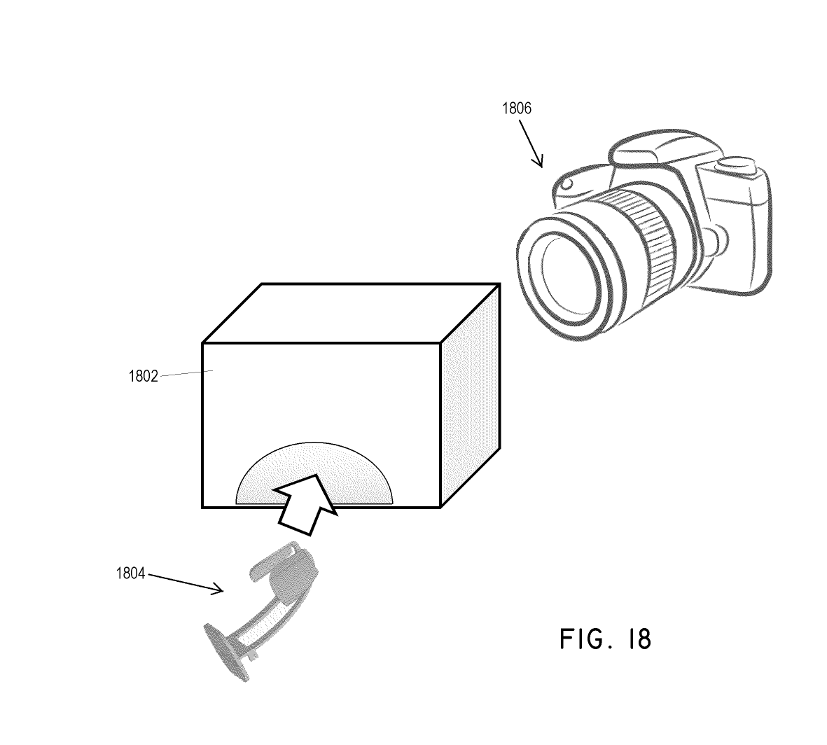

In some embodiments, as shown in FIG. 18, before each session of imaging with a patient using a particular set of camera 1806 and illuminator 1804, an image of an optically diffuse material (foam or optical rubber phantom) 1802 can be captured using either in transmission geometry or reflectance geometry. The diffuse material 1802 can be made of various light diffusion objects such as ABS foam, Styrofoam, or silicone rubber and can be used with scattering materials. The diffuse material 1802 can be formed into any shape, and the general shape is not limiting. The illuminator 1804 having, for example, LEDs can be inserted into a cavity that can be designed to simulate the curvature of the oral upper palate. The camera 1806 can then be used to capture an image from any side of the material 1802 such as, for example, the opposite side of the illuminator 1804. Using this intensity balancing images from different sessions or different cameras, average intensity or specific intensity per pixels can then be compared to generate relative correction factor for the patient images. This intensity balancing procedure can be advantageous for achieving quantitative comparison between individuals as well as comparing longitudinal changes of a single individual. A standardized intensity balancing pad can be built from both ABS foam as well as optically-stable silicone rubber blocks mixed with strong optical scatterers such as TiO₂ and absorbing materials to further attenuate the light intensity over a wide range of wavelengths.

### EXAMPLES

### Clinical Study I

77 patients were tested, and among them 45 have been confirmed CT images. The others were presented with possible CT images at a later date or CT images have not been entered into the UCI's patient records. This lack of CTs on some patients can be attributed to the fact the CTs are not always taken at the ENT specialist's location. Oftentimes the patient has long history of sinus-related problems and have had CTs taken for various reasons. Unless there is an urgent need by the attending physician, additional CT may not be taken. However, NIR images were acquired with the anticipation that the CTs could be acquired for most if not all patients. 45 cases with CTs are composed of 13 healthy cases, 7 mild cases and 25 severe cases.

Table I summarizes the results of the diagnoses based on three types of evidence independently: symptoms, NIR intensity alone, and NIR intensity with pattern. The CT images were used as the gold standard for the actual diagnosis for each case. CT images were categorized into healthy if the cavity is empty, mild if mucosal thickening is present, and severe if mucosal thickening and fluid present occupies more than ¾ of the sinus cavities. Symptoms were categorized into healthy/mild/severe cases. NIR images were categorized into normal, mild and severe based on NIR intensity only, or NIR pattern and intensity combined. As an initial test, qualitative criteria (i.e. pattern recognition of 2 operators) were used for NIR-based diagnosis. More systematic approach for the image pattern and intensity will be performed in the next 6 months. The sensitivity and specificity were then calculated for diagnosis between healthy and sinusitis (which includes both mild and severe cases). Results are presented in the second third rows in the table and they indicate that the NIR images do provide comparable performance as the symptoms as diagnostic evidence for determining presence of some type of disease. However, in reality, NIR images will be in conjunction with symptoms, not separately. In some embodiments, sensitivity and specificity may be beyond 70%.

TABLE I
Diagnosis Table

| | | Symptoms | NIR Intensity | |
|---|---|---|---|---|
| | | vs. | vs. | NIR Pattern & |
| | | CT | CT | Intensity vs. CT |
| Diagnosis | Sensitivity | 69% | 70% | 69% |
| (Healthy vs | Specificity | 75% | 63% | 56% |
| Sinusitis) | | | | |
| Differential | Sensitivity | 75% | 83% | 95% |
| (Severe vs. | Specificity | 56% | 43% | 55% |
| Mild) | | | | |

In the bottom two rows, the sensitivity and specificity are presented for differential diagnosis between mild and severe cases. Sensitivity and specificity are statistical measures of the performance of a binary classification test. Sensitivity measures the probability of a positive test result being correct (the percentage of diseased patients who have been correctly diagnosed). Specificity measures the probability of a negative test result being correct. The combination of the NIR pattern and intensity as the only diagnostic criteria achieves 95% sensitivity, which is substantially better than symptoms or NIR intensity alone. On the other hand the specificity still remains below the 75%. The results above reflect the worst possible cases for the performance of NIR images.

### Clinical Study II

NIR imaging of patients with confirmed sinusitis was performed and compared with computed tomography (CT) scans. Comparison between CT scans and NIR image patterns demonstrates correlation between the NIR image intensity and the frontal bone structures of the maxillary sinuses. Results from the patient study show that air-filled and fluid/tissue-filled spaces can be reasonably distinguished by their differing NIR signal penetration patterns as well as reduced transmittance of NIR light by fluid build-up.

In order to better understand the sources of the light patterns, an algorithm was developed for volume segmentation of the CT images based on the intensity for bone, soft tissue and cavities. In some embodiments, a segmentation algorithm can be used.

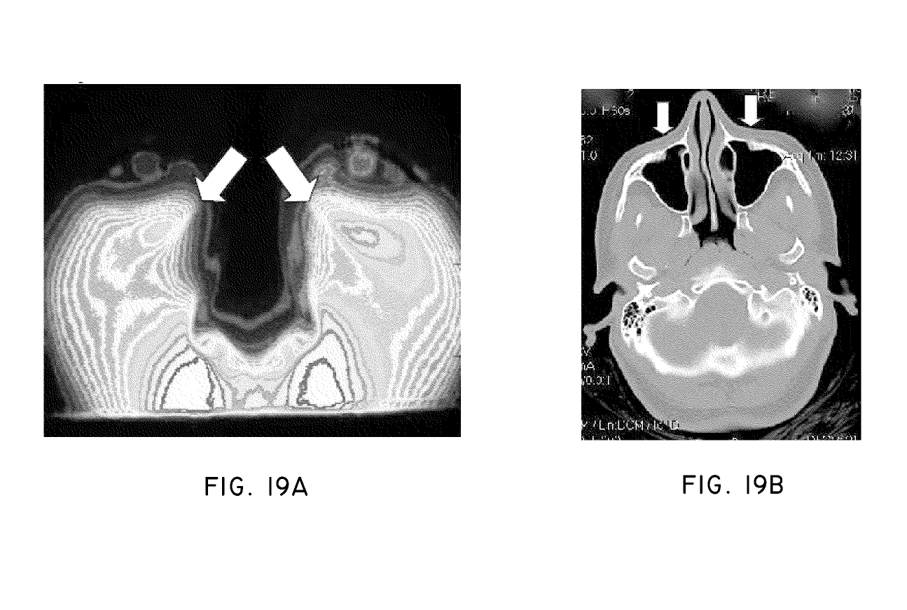

10 patients were collected with CT scans and/or other confirmed sinus infections (i.e. via endoscopic examination). In total, one normal subject was enrolled with confirmed ‘clear’ CT image of both sinuses—the control subject—and 10 sinus infection patients with CT scans. Comparing NIR image and CT image seems to provide clues to a possible correlation between NIR patterns and facial structure. For example, the high NIR intensity peaks below the eyeballs in normal subjects. An examination of the CT scan reveals a thin tissue structures around the same region. This is illustrated in the FIGS. 19A-B. As shown in FIGS. 19A-B, intensity peaks below eyes correspond to the thin cheekbone region as shown in the CT.

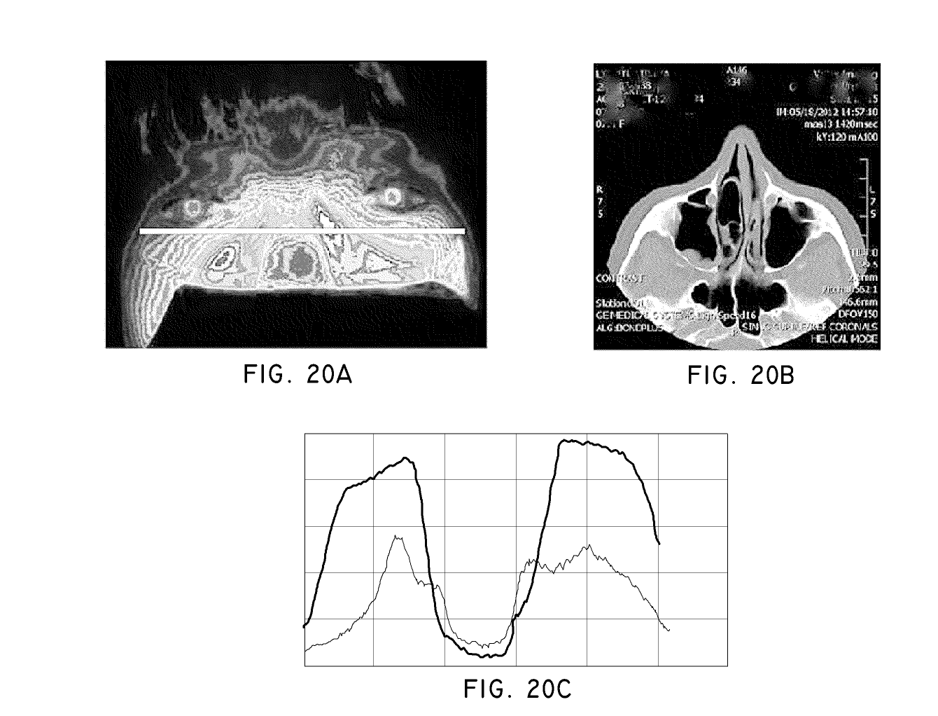

Typically, reduced intensity of the NIR transmittance and/or severe asymmetry between and left and right sinus transmittance can indicate sinus infections. The following figures highlight these findings in qualitative fashion, but provide some indications whether the NIR imaging may be useful as a quantitative tool. FIGS. 20A-C illustrate the case of moderate sinus infection and moderately reduced NIR intensity. FIG. 20A shows an NIR image with contour map. FIG. 20B shows a CD of the patient illustrating the moderate mucous buildup inside the maxillary sinuses. FIG. 20C shows a lateral NIR line intensity profile along a horizontal line (shown) below the eyes. The X axis of FIG. 20C is the pixel position along the horizontal line. The Y axis is the intensity profile from a known health patient. The bold line shows the line intensity profile from a known healthy patient. The non-bold line shows the line intensity profile from a patient diagnosed with mild sinusitis. The changes in the NIR patterns and moderate reduction in the NIR intensity are notable.

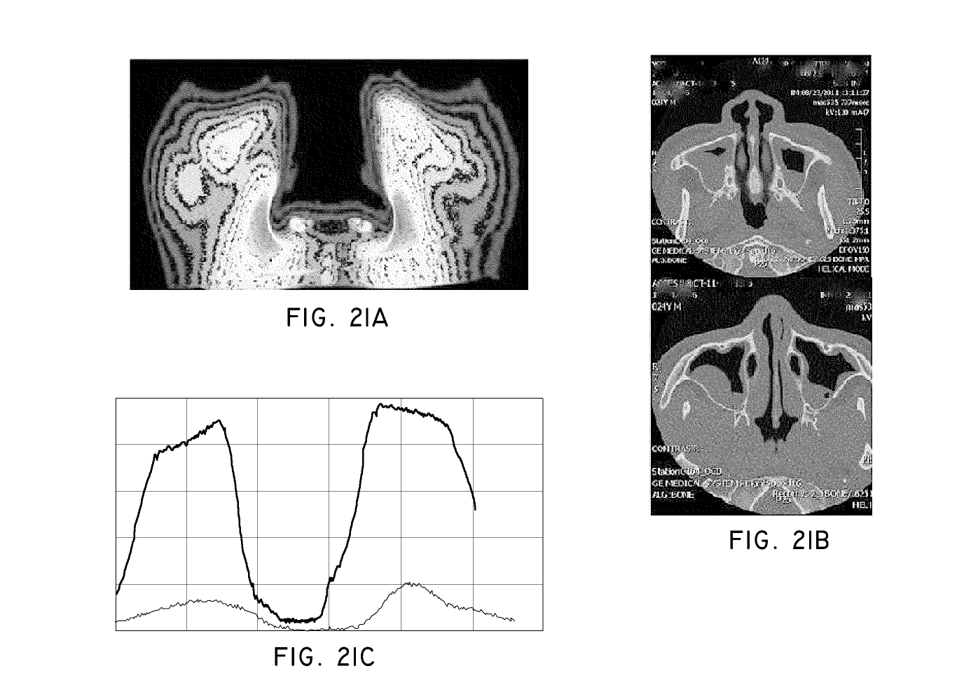

FIGS. 21A-C illustrate the case of severe sinus infection and very low NIR transmittance. Mucosal thickening is visibly notable in the CT scans and reduction in NIR intensity is much more drastic in comparison to the moderate case above in the FIGS. 18A-C.

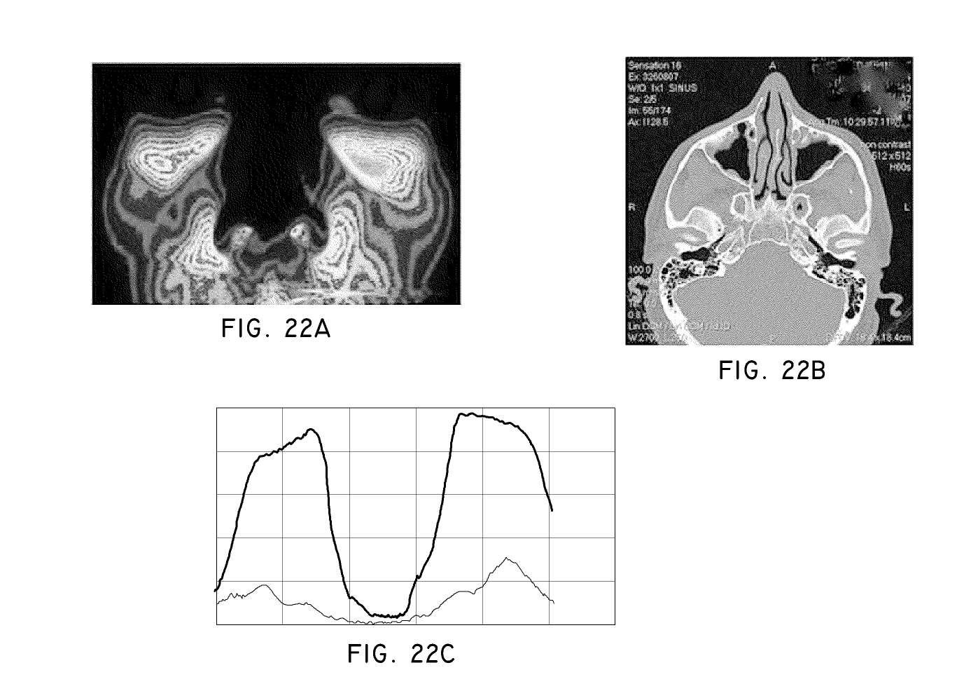

FIGS. 22A-C illustrate another case of severe sinus infection but only moderate mucosal thickening is present in the CT scans. This patient has just undergone sinus surgery and is due for a follow up in 2-3 weeks from the date of this report.

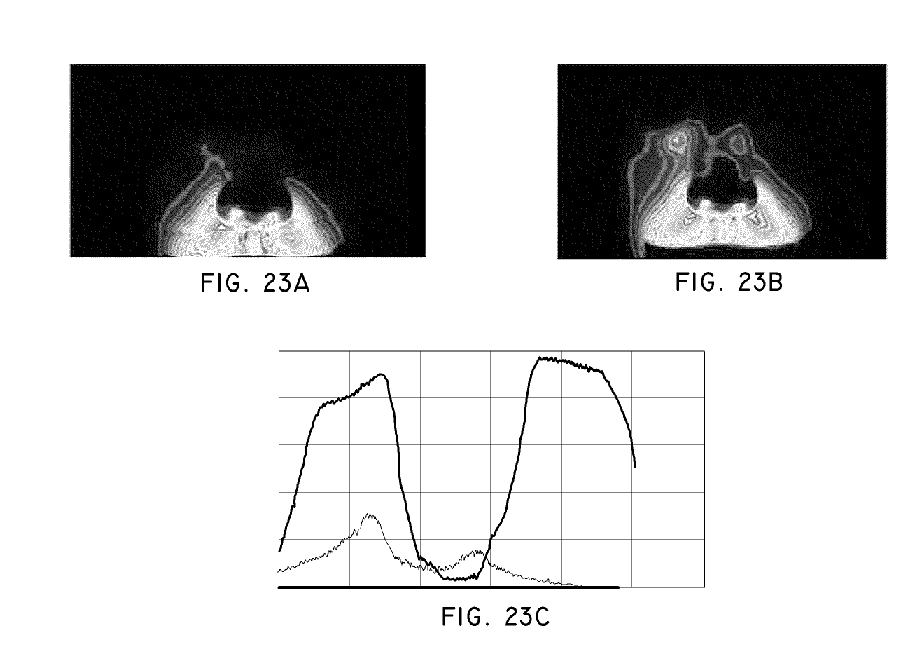

FIGS. 23A-C show NIR images of a patient who just had a surgery 2 weeks ago and had debridement procedure (suctioning out fluid built up days after surgery) done on the day of NIR imaging. Images for before and images for after debridement show quite small contrast in terms of the pattern alone. However, when the line plot is drawn along the peaks, the contrast is enhanced much strong. The patient's NIR transmittance has not fully recovered to normal level even after debridement, which may be due to the healing process (i.e. swelling or incomplete removal of fluid).

Although the foregoing description has shown, described, and pointed out the fundamental novel features of the present teachings, it will be understood that various omissions, substitutions, and changes in the form of the detail of the apparatus as illustrated, as well as the uses thereof, may be made by those skilled in the art, without departing from the scope of the present teachings. Consequently, the scope of the present teachings should not be limited to the foregoing discussion, but should be defined by the appended claims.

What is claimed is:

1. A device for imaging a sinus cavity of a patient, the device comprising:
a flexible illuminator configured to conform to the patient's upper palate;
an optical output located on a distal end of the flexible illuminator, wherein the optical output is configured to direct radiation towards the patient's sinus cavity;
an optical detector configured to receive the radiation from the optical output that passes through the patient's sinuses and out the patient's tissue, and provide a output signal; and
an analyzer configured to create an iso-intensity line map from the output signal.

2. The device of claim 1, wherein the radiation is near-infrared light.

3. The device of claim 2, wherein the near-infrared light has a wavelength of about 850 nm.

4. The device of claim 1, wherein the optical detector is a camera.

5. The device of claim 1, wherein the device further comprises an optical diffusion layer located between the optical output and the patient's upper palate.

6. The device of claim 5, wherein the optical diffusion layer comprises a light scattering foam.

7. The device of claim 1, wherein the optical output is a plurality of LEDs.

8. The device of claim 7, wherein the plurality of LEDs are arranged in a bifurcated array.

9. The device of claim 1, wherein the analyzer can determine absolute light intensity levels along peak regions of iso-intensity lines, average intensity between a patients left and right sinuses, and asymmetry between left and right sinuses.

10. A method of imaging a sinus cavity of a patient, the method comprising:
placing a flexible illuminator into the patient's mouth, wherein the flexible illuminator is configured to conform to the patient's upper palate;
irradiating the patient's sinuses using optical sources located on a distal end of the flexible illuminator;
receiving the radiation that passes through the patient's sinuses and out the patient's tissue; and
analyzing the received radiation by determining an iso-intensity line map from the received radiation.

11. The method of claim 10, wherein the flexible illuminator further comprises an optical diffusion layer located between the optical sources and the patient's upper palate.

12. The method of claim 11, wherein the optical diffusion layer comprises a light scattering foam.

13. The method of claim 10, wherein the optical sources are a plurality of LEDs.

14. The method of claim 13, wherein the plurality of LEDs are arranged in a bifurcated array.

15. An at home self-diagnostic system for imaging a patient's sinus cavity, the system comprising:
a stand configured to receive the patient's face so that the patient's face remains in a generally fixed position;
a flexible illuminator configure to conform to the patient's upper palate;
an optical output located on a distal end of the flexible illuminator, wherein the optical output is configured to direct radiation towards the patient's sinus cavity;
an activator configured to activate the optical output;
an optical detector located at a position to receive the radiation from the optical output that passes through the patient's sinuses and out the patient's tissue, and provide a output signal; and
an analyzer configured to create an iso-intensity map from the output signal.

16. The self-diagnostic system of claim 15, wherein the system further comprises a display to display the iso-intensity map.

17. The self-diagnostic system of claim 15, wherein the system further comprises an optical diffusion layer located between the optical output and the patient's upper palate.

18. The self-diagnostic system of claim 17, wherein the optical diffusion layer comprises a light scattering foam.

19. The self-diagnostic system of claim 15, wherein the optical output is a plurality of LEDs.

20. The self-diagnostic system of claim 19, wherein the plurality of LEDS are arranged in a bifurcated array.
